(19) United States
(12) Patent Application Publication
(10) Pub. No.: US 2022/0047322 A1
(43) Pub. Date: Feb. 17, 2022

(54) CORING AND AMPUTATION DEVICES, SYSTEMS, AND METHODS

(71) Applicant: Ethicon, Inc., Somerville, NJ (US)

(72) Inventors: William Cohn, Houston, TX (US); Terry Daglow, Houston, TX (US); Matthew Kuhn, Houston, TX (US); Jonathan Melchor, Warwick, RI (US); Theodore Mick, Houston, TX (US); Chris Mills, Houston, TX (US); Steven Nguyen, Cypress, TX (US); Jorge Salazar, Houston, TX (US)

(21) Appl. No.: 17/381,378

(22) Filed: Jul. 21, 2021

Related U.S. Application Data
(60) Provisional application No. 63/066,457, filed on Aug. 17, 2020.

(51) Int. Cl. A61B 18/14

(52) U.S. Cl. CPC A61B 18/149; A61B 2018/0063

(57) ABSTRACT

A method for removing tissues may comprise disposing a tissue resection device at a target tissue site, causing the tissue resection device to resect a core of tissue from the target tissue site, removing the core of tissue from the body, wherein the removing the core of tissue from the body creates a core cavity at the target tissue site.

### CROSS-REFERENCE TO RELATED APPLICATIONS

This application claims priority to and the benefit of U.S. Patent Application No. 63/066,457 filed Aug. 17, 2020, which is hereby incorporated by reference in its entirety.

### BACKGROUND

Cancer is not a single disease, but rather a collection of related diseases that can start essentially anywhere in the body. Common amongst all types of cancer is that the body's cells begin to divide without stopping, proliferating, and potentially spreading into surrounding tissues. In the normal course of events, cells grow and divide to form new cells as required by the body and when they become damaged or old, they die, and new cells replace the damaged or old cells; however, cancer interrupts this process. With cancer, the cells become abnormal, and cells that should die do not and new cells form when they are not needed. These new cells can reproduce or proliferate without stopping and may form growths called tumors.

Cancerous tumors are malignant, which means they can spread into or invade surrounding healthy tissue. In addition, cancer cells can break off and travel to remote areas in the body through blood or in the lymph system. Benign tumors, unlike malignant tumors, do not spread or invade surrounding tissue; however, they may grow large and cause damage. Both malignant and benign tumors may be removed or treated. Malignant tumors tend to grow back whereas benign tumors can grow back but are much less likely to do so.

Cancer is a genetic disease in that it is caused by changes in the genes that control the ways that cells function, especially in how they grow and divide. Genetic changes that cause cancer may be inherited or they may arise over an individual's lifetime as a result of errors that occur as cells divide or because of damage to DNA caused by certain environmental exposure, for example, industrial/commercial chemicals and ultraviolet light. The genetic changes that may cause cancer tend to affect three types of genes; namely proto-oncogenes which are involved in normal cell growth and division, tumor suppressor genes which are also involved in controlling cell growth and division, and DNA repair genes which, as the name implies, are involved in repairing damaged DNA.

More than one-hundred distinct types of cancer have been identified. The type of cancer may be named for the organ or tissue where the cancers arise, for example, lung cancer, or the type of cell that formed them, for example squamous cell cancer. Cancer, unfortunately, is a leading cause of death both in the United States and world-wide. According to the World Health Organization, the number of new cancer cases will rise to twenty-five (25) million per year over the next two decades.

Lung cancer is one of the most common cancers today. According to the World Cancer Report 2014 from the World Health Organization, lung cancer occurred in 14 million people and resulted in 8.8 million deaths world-wide, making it the most common cause of cancer-related death in men and the second most common cause of cancer-related death in women. Lung cancer or lung carcinoma is a malignant lung tumor that if left untreated can metastasize into neighboring tissues and organs. The majority of lung cancer is caused by long-term tobacco smoking; however, about 10 to 15 percent of lung cancer cases are not tobacco related. These non-tobacco cases are most often caused by a combination of genetic factors and exposure to certain environmental conditions, including radon gas, asbestos, second-hand tobacco smoke, other forms of air pollution, and other agents. The chance of surviving lung cancer as well as other forms of cancer depends on early detection and treatment.

Improvements in removing tissue are needed.

### SUMMARY

It may be desirable to remove a core of tissue from other target tissue sites including, but not limited to, the lungs, the liver, pancreas, or gastrointestinal (GI) tract, for which managing post-coring bleeding may be desired. A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

A method for coring tissue may comprise disposing a tissue resection device at a target tissue site, causing the tissue resection device to resect a core of tissue from the target tissue site, and removing the core of tissue from the body, wherein the removing the core of tissue from the body creates a core cavity at the target tissue site. The core of tissue comprises at least a portion of a tissue lesion. The resecting the core of tissue from the target tissue site may comprise mechanical transection. The resecting the core of tissue from the target tissue site may comprise the delivery of radiofrequency energy. The resecting the core of tissue from the target tissue site may comprise mechanical compression and the delivery of radiofrequency energy. The resecting the core of tissue from the target tissue site may comprise transection with an energized wire. The resecting the core of tissue from the target tissue site may comprise one of more of mechanical compression, the delivery of radiofrequency energy, the delivery of microwave energy, the delivery of ultrasonic energy, or transection with an energized wire. Other resection devices and procedures may be used. The resection device may be configured for one or more of mechanical compression, the delivery of radiofrequency energy, the delivery of microwave energy, the delivery of ultrasonic energy, or transection with an energized wire.

Methods for coring tissue may further comprise inserting a sleeve into the core cavity to support a wall of the core cavity. Methods for coring tissue may further comprise delivering radiofrequency energy to at least a portion of a wall defining the core cavity. Methods for coring tissue may further comprise delivering chemotherapy to at least a portion of a wall defining the core cavity. Methods for coring tissue may further comprise delivering microwave energy to at least a portion of a wall defining the core cavity. Methods for coring tissue may further comprise delivering thermal energy to at least a portion of a wall defining the core cavity. Methods for coring tissue may further comprise delivering ultrasonic energy to at least a portion of a wall defining the core cavity.

Methods for coring tissue may further comprise sealing biological fluid vessels. The sealing biological fluid vessels may minimize flow of biological fluids into the cavity core. The sealing may be effected using at least mechanical compression. The sealing may be effected using at least radiofrequency energy. The sealing may be effected using at least microwave energy. The sealing may be effected using at least ultrasonic energy. The sealing may be effected using one or more of compression or delivery of energy such as radiofrequency, microwave, ultrasonic, or thermal energy.

The present disclosure relates to a system, device, and method for performing lung lesion removal. A lung needle biopsy is typically performed when an abnormality is found on an imaging test, for example, an X-ray or CAT scan. In a lung needle biopsy, a fine needle is used to remove sample of lung tissue for examining under a microscope to determine the presence of abnormal cells. Tissue diagnosis is challenging in small (<6 mm) and intermediate (6-12 mm) nodules. CT guided biopsy of peripheral lesions, either through the chest wall (80%) or by means of a bronchoscope (20%) yields only a 0.001-0.002 cm2 of diagnostic tissue, and as such, cancer, when present, is only successfully identified in 60% of small and intermediate nodules. Although bronchoscopic techniques and technology continue to evolve, biopsy accuracy, specificity, and sensitivity will always be limited when dealing with small and intermediate nodules in the periphery of the lung.

If it is determined that the lesion is cancerous, a second procedure may be performed to remove the lesion and then followed up with chemotherapy and/or radiation. The second procedure most likely involves lung surgery. These procedures are typically done through an incision between the ribs. There are a number of possible procedures depending on the state of the cancer. Video-assisted thoracic surgery is a less invasive procedure for certain types of lung cancer. It is performed through small incisions utilizing an endoscopic approach and is typically utilized for performing wedge resections of smaller lesions close to the surface of a lung. In a wedge resection, a portion of the lobe is removed. In a sleeve resection, a portion of a large airway is removed thereby preserving more lung function.

Nodules deeper than 2-3 cm from the lung surface, once identified as suspicious for cancer, are difficult to localize and excise using laparoscopic or robotic lung sparing technique despite pre-procedure image guided biopsy and localization. Thus, surgeons perform an open thoracotomy or lobectomy to remove lung nodules that are 2-3 cm from the lung surface. A thoracotomy is an open approach surgery in which a portion of a lobe, a full lobe or an entire lung is removed. In a pneumonectomy, an entire lung is removed. This type of surgery is obviously the most aggressive. In a lobectomy, an entire section or lobe of a lung is removed and represents a less aggressive approach than removing the entire lung. All thoracoscopic lung surgeries require trained and experienced thoracic surgeons and the favorability of surgical outcomes track with operative experience.

Any of these types of lung surgery is a major operation with possible complications which depend on the extent of the surgery as well as the patient's overall health. In addition to the reduction in lung function associated with any of these procedures, the recovery may take from weeks to months. With a thoracotomy, spreading of the ribs is required, thereby increasing postoperative pain. Although video-assisted thoracic surgery is less invasive, there can still be a substantial recovery period. In addition, once the surgery is complete, full treatment may require a system chemotherapy and/or radiation treatment.

As set forth above, a fine needle biopsy may not prove to be totally diagnostic. The fine needle biopsy procedure involves guiding a needle in three-dimensional space under two-dimensional imaging. Accordingly, the doctor may miss the lesion, or even if he or she hits the correct target, the section of the lesion that is removed through the needle may not contain the cancerous cells or the cells necessary to assess the aggressiveness of the tumor. A needle biopsy removes enough tissue to create a smear on a slide. The device of the present disclosure is designed to remove the entire lesion, or a substantial portion of it, while minimizing the amount of healthy lung tissue removal. This offers a number of advantages. Firstly, the entire lesion may be examined for a more accurate diagnosis without confounding sampling error, loss of cell packing or gross architecture. Secondly, since the entire lesion is removed, a secondary procedure as described above may not be required. Thirdly, localized chemotherapy and/or energy-based tumor extirpation, such as radiation, may be introduced via the cavity created by the lesion removal.

In at least one embodiment, the disclosure defines a method for removing a tissue lesion including anchoring to the tissue lesion; creating a channel in the tissue leading to the tissue lesion; creating a tissue core including the tissue lesion; ligating the tissue core at a ligation point downstream from the tissue lesion; amputating the tissue core form the tissue between the ligation point and the tissue lesion; and removing the tissue core from the channel.

In keeping with aspects of the disclosure, the sleeve may be inserted in the channel prior to or after removing the tissue core. The sleeve may also be anchored to the tissue. In keeping with another aspect of the disclosure, a localized treatment may be delivered through the sleeve.

In some embodiments, creating a tissue core includes cauterizing and cutting tissue. Ligating tissue may include tissue may include cauterizing tissue at a specific location known as the ligation point. Amputation of the tissue core may be performed with a snare, an energized wire, or any other device capable of slicing tissue.

In some embodiments, the tissue core is created by first sealing blood vessels then slicing tissue to form the core.

### BRIEF DESCRIPTION OF THE DRAWINGS

The following drawings show generally, by way of example, but not by way of limitation, various examples discussed in the present disclosure. In the drawings:

FIG. 1 depicts a tissue resection device in accordance with an embodiment of the present disclosure.

FIG. 2 illustrates a sectional view of the tissue resection device of FIG. 1.

FIG. 3 shows a sectional view of a tissue resection device in accordance with an embodiment of the present disclosure.

FIG. 4 depicts a sectional view of a tissue resection device in accordance with an embodiment of the present disclosure.

FIG. 5 illustrates an exemplary anchor that may be employed in a lesion removal method in accordance with an embodiment of the present disclosure.

FIG. 6 shows a series of incision blades for use in a lesion removal method in accordance with an embodiment of the present disclosure.

FIG. 7 displays tissue dilators suitable for use in a lesion removal method in accordance with an embodiment of the present disclosure.

FIG. 8 shows a flow diagram of an example method for coring and for sealing tissue.

FIGS. 9A-9B illustrate an example trocar.

FIG. 10 illustrates an example trocar.

FIG. 11 illustrates an example bipolar coring device.

FIG. 12 illustrates an example bipolar coring device.

FIG. 13 illustrates an example bipolar amputation device.

FIG. 14 illustrates an example bipolar amputation device.

FIG. 15 depicts details of the tissue resection device in accordance with an embodiment of the present disclosure.

FIG. 16 depicts details of the device with the cutting tube withdrawn to reveal details of the grooves and snares.

### DETAILED DESCRIPTION

The present disclosure relates to systems and methods for coring tissue. Various tissue and sites may benefit from the disclosed systems and methods.

A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions.

The present disclosure relates to methods and systems for coring tissue. Methods for coring tissue may comprise disposing a tissue resection device at a target tissue site, causing the tissue resection device to resect a core of tissue from the target tissue site, and removing the core of tissue from the body, wherein the removing the core of tissue from the body creates a core cavity at the target tissue site. The core of tissue comprises at least a portion of a tissue lesion. The resecting the core of tissue from the target tissue site may comprise mechanical transection. The resecting the core of tissue from the target tissue site may comprise the delivery of radiofrequency energy. The resecting the core of tissue from the target tissue site may comprise mechanical compression and the delivery of radiofrequency energy. The resecting the core of tissue from the target tissue site may comprise transection with an energized wire. The resecting the core of tissue from the target tissue site may comprise one of more of mechanical compression, the delivery of radiofrequency energy, the delivery of microwave energy, the delivery of ultrasonic energy, or transection with an energized wire. Other resection devices and procedures may be used. The resection device may be configured for one or more of mechanical compression, the delivery of radiofrequency energy, the delivery of microwave energy, the delivery of ultrasonic energy, or transection with an energized wire.

The present disclosure relates to methods and systems for coring tissue and sealing the core cavity created by removing the tissue core. Such methods may comprise disposing a fill material in the core cavity. Methods may comprise applying pressure to a portion of the core cavity such as to a wall defining the core cavity. Methods may comprise ablating a portion of the core cavity such as a wall defining the core cavity. Methods may comprise causing a cavity closure device, such as suture thread, a stapling device, an ultrasonic tissue sealing device, a bipolar radiofrequency sealing device, or any combination thereof to close the tissue cavity. Methods may comprise disposing a cavity sealing material, such as a tissue graft, a hemostatic patch, a hemostatic agent such as fibrin or thrombin, a biological adhesive material such as Dermabond®, or any combination thereof to close the tissue cavity.

Methods may comprise coring and sealing blood vessel simultaneously as coring procedure is implemented. For example, radiofrequency energy may be provided between the coil and anvil electrodes. As a further example, a coil may be rotated into a target site, an anvil electrode may be cause to close against the coil, a radiofrequency energy may be used to seal areas adjacent the target site, and tissue may be cored using a cutting blade. Such a sequence may be repeated until the cored tissue is within a cutting tube. At this point, ligation (e.g., with another set of electrodes) may be performed to seal any potential blood vessel connecting the cored tissue with surrounding tissue. In an aspect, a mechanical ligation line may be deployed to finish the coring process so that cored tissue can be removed, leaving the cored cavity ready for any subsequent step.

Methods may comprise any combination or permutation of: 1) disposing an anchoring device into a tissue cavity, 2) disposing a tissue access port into the tissue cavity, 3) disposing a tissue sealing device into the tissue cavity (with or without a tissue access port, with or without guidance from an anchoring device), 4) causing the tissue sealing device to seal at least a portion of the tissue cavity, 5) introducing a fill material into the tissue cavity (with or without a fill material delivery device, with or without being preceded by disposing a tissue sealing device into the tissue cavity, with or without removing the tissue sealing device after sealing at least a portion of the tissue cavity, with or without a tissue access port), 6) disposing a cavity sealing material adjacent to the tissue cavity (with or without being preceded by disposing a tissue sealing device into the tissue cavity, with or without removing the tissue sealing device after sealing at least a portion of the tissue cavity, with or without being preceded by introducing a fill material into the tissue cavity), 7) disposing a cavity closure device adjacent to the tissue, and 8) causing a cavity closure device to close the tissue cavity (with or without being preceded by any combination or permutation of the above steps). As described herein, methods may be used to core and/or seal tissue at various target sites. Although a lung is used as an illustrative example, it should not be so limiting, as other target sites may be punctured or actively cored and may benefit from the disclosed sealing methods.

Various methods, devices, and systems may be used to core or remove tissue.

A method for removing a tissue lesion may comprise introducing a tissue resection device to a target tissue site, causing the tissue resection device to resect a core of tissue from the target tissue site, and removing the core of tissue from the body. The core of tissue may comprise at least a portion of a tissue lesion. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may comprise mechanical compression and the delivery of radiofrequency energy. A method may further comprise amputating the core of tissue from the target tissue site. As an example, the means for amputation of the core of tissue may comprise mechanical transection. As a further example, the means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire. Other devices may be used.

A method for removing a core of tissue may comprise introducing a tissue resection device to a target tissue site, causing the tissue resection device to resect a core of tissue from the target tissue site, and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for the delivery of radiofrequency energy. The tissue resection device may be configured for mechanical transection. The tissue resection device may be configured for mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for removing a core of tissue may comprise introducing a tissue resection device to a target tissue site. The tissue resection device may comprise one or more of: a first clamping element comprising a helical coil and a first electrode, or a second clamping element comprising a second electrode. Where a second clamping element is included, the second clamping element may be positioned to oppose at least a portion of the first clamping element. The method may further comprise causing the tissue resection device to resect a core of tissue from the target tissue site and removing the core of tissue from the body. A method may further comprise creating a core cavity at the target tissue site. A method may further comprise inserting a sleeve into the core cavity. A method may further comprise delivering radiofrequency energy through the core cavity. A method may further comprise delivering chemotherapy through the core cavity. A method may further comprise delivering microwave radiation through the core cavity. A method may further comprise delivering thermal energy through the core cavity. A method may further comprise delivering ultrasonic energy through the core cavity. The tissue resection device may be configured for resecting the core of tissue comprises the delivery of radiofrequency energy. The tissue resection device may be configured for resecting the core of tissue comprises mechanical transection. The tissue resection device may be configured for resecting the core of tissue comprises mechanical compression and the delivery of radiofrequency energy. A method may further comprise amputating the core of tissue from the target tissue site. The means for amputation of the core of tissue may comprise mechanical transection. The means for amputation of the core of tissue may comprise the delivery of radiofrequency energy. The means for amputation of the core of tissue may comprise mechanical compression and the delivery of radiofrequency energy. The means for amputation of the core of tissue may comprise transection with an energized wire.

A method for sealing biological fluid vessels may comprise piercing a target tissue site containing a least a portion of at least one target biological fluid vessel with a helical tissue sealing mechanism, wherein the helical tissue sealing mechanism comprises: a helical piercing element and a clamping element. Wherein the method may comprise causing the helical tissue sealing mechanism to apply mechanical compression to at least one target biological fluid vessel and delivering energy to seal at least one target biological fluid vessel. The helical piercing element may comprise the clamping element. The mechanical compression may be applied between the helical piercing element and the clamping element. A method may further comprise a second clamping element. The mechanical compression may be applied between the first and second clamping elements. The delivered energy may comprise monopolar radiofrequency energy. The delivered energy may comprise bipolar radiofrequency energy. The delivered energy may comprise thermal energy. The delivered energy comprises ultrasonic energy.

A method for sealing biological fluid vessels may comprise piercing a target tissue site with a helical piercing element, adjusting the pitch of the helical piercing element to apply mechanical compression to the target tissue, and delivering energy to seal at least one biological fluid vessel in the target tissue. The helical piercing element may comprise a plurality of tissue sealing electrodes. The delivered energy may comprise monopolar radiofrequency energy. The delivered energy may comprise bipolar radiofrequency energy. The delivered energy may comprise thermal energy. The delivered energy may comprise ultrasonic energy.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. A tissue resection device may further comprise: a first actuator operable to actuate the first or second clamping element to apply mechanical compression to tissue and a second actuator operable to actuate the cutting element to transect tissue. The helical coil may include first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the first clamping element. The second electrode may be comprised by at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element having a helical coil disposed on a distal end, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment comprises a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the helical coil. The first electrode may be comprised by at least a portion of the first clamping element. The second electrode may be comprised by at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil and a first electrode, and a second clamping element comprising a second electrode, the second clamping element being positioned to oppose at least a portion of the first clamping element. The first and second clamping elements may be configured for: (a) the delivery of radiofrequency energy for sealing tissue, and (b) the application of mechanical compression for the transection of tissue. The tissue resection device may further comprise a first actuator operable to actuate the first or second clamping element to apply mechanical compression to tissue and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the helical coil. The first electrode may be comprised by at least a portion of the first clamping element. The second electrode may be comprised by at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A surgical instrument system for the resection of tissue may comprise an end effector operable to cut and seal tissue, wherein the end effector and a generator configured to provide power to the end effector having the first and second electrodes for sealing tissue. The end effector may comprise a first clamping element comprising a helical coil, a second clamping element, the second clamping element being positioned to oppose at least a portion of the first clamping element, a first and second electrode configured for the delivery of radiofrequency energy for sealing tissue, and a cutting element configured for the transection of at least a portion of the sealed tissue. The surgical instrument system may further comprise a controller in communication with the generator, wherein the controller is configured to control the generator to provide radiofrequency energy sufficient to seal tissue to the first and second electrodes of the end effector, based on at least one sensed operating condition of the end effector. The controller may be configured to sense the presence of tissue at the end effector. The controller may be configured to sense the presence of tissue at the end effector based on a measured impedance level associated with the first and second electrodes. The controller may be configured to sense an amount of force applied to at least one of the first or second clamping elements to detect the presence of tissue at the end effector. The controller may be configured to sense the position of the cutting element relative to at least one of the first or second clamping elements. The controller may be configured to control the generator to provide radiofrequency energy at the end effector when the second actuator is actuated and no tissue is sensed at the end effector. The controller may be configured to control the generator to provide a continuous amount of radiofrequency energy. The controller may be configured to control the generator to automatically provide an increase or decrease in the amount of radiofrequency energy. The system may further comprise: a first actuator operable to actuate the first or second clamping element to apply mechanical compression to tissue; and; a second actuator operable to actuate the cutting element to transect tissue. The helical coil may comprise first and second contiguous coil segments, the first coil segment including the first electrode. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the helical coil. The first electrode may be comprised by at least a portion of the first clamping element. The second electrode may be comprised by at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue resection apparatus may comprise a first clamping element comprising a helical coil, a second clamping element, the second clamping element being positioned to oppose at least a portion of the first clamping element, a first and second electrode configured for the delivery of radiofrequency energy for sealing tissue, a first cutting element configured for the transection of at least a portion of the sealed tissue, a first and second ligating element, and a second cutting element positioned between said first and second ligating elements. The tissue resection device may further comprise a first actuator operable to actuate the first or second clamping element to apply mechanical compression to tissue, and a second actuator operable to actuate the cutting element to transect tissue. The helical coil may comprise first and second contiguous coil segments. The first coil segment may comprise a generally planar open ring. The first coil segment may be helical and may have a pitch of zero. The second coil segment may be helical and may have a non-zero pitch. The second coil segment may have a variable pitch. The first coil segment may be helical and may have a first pitch and the second coil segment may be helical and may have a second pitch, and at least one of the first and second pitches may be variable. The first electrode may be comprised by at least a portion of the helical coil. The first electrode may be comprised by at least a portion of the first clamping element. The second electrode may be comprised by at least a portion of the second clamping element. The helical coil may comprise a blunt tip. The first and second electrodes may comprise surface profiles that are matching or substantially matching. At least a portion of the cutting element may comprise a sharpened edge. The cutting element may comprise at least one electrode configured for the delivery of radiofrequency energy. The cutting element may comprise an ultrasonic blade. The tissue resection device may further comprise a second cutting element configured for the amputation the core of tissue from the target tissue site. At least a portion of the second cutting element may comprise a sharpened edge. The second cutting element may comprise at least one electrode configured for the delivery of radiofrequency energy. The second cutting element may comprise an energized wire. The second cutting element may comprise a suture. The tissue resection device may further comprise an actuator operable to actuate the second cutting element to transect tissue.

A tissue sealing mechanism may comprise a helical coil with a generally obround cross section and a tapered point disposed at a distal end, a first and second helical tissue sealing surface, wherein the first and second helical tissue sealing surfaces are provided by the parallel planar surfaces of the helical coil, a first electrode disposed on the first helical tissue sealing surface, and a second electrode disposed on the second helical tissue sealing surface, wherein the first and second electrodes are configured to apply bipolar radiofrequency energy for sealing tissue. The helical coil may comprise first and second contiguous coil segments. The helical coil may comprise a blunt tip. The first and second electrodes may have surface profiles that are substantially matching. The first and second helical tissue sealing surfaces may further comprise a plurality of electrodes configured for the delivery of bipolar radiofrequency energy.

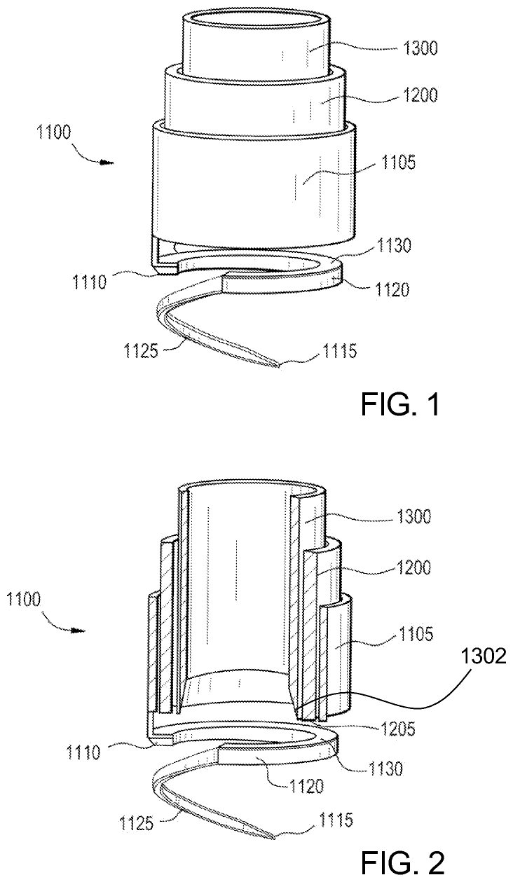

FIGS. 1-7 show examples devices that may be used to effect a coring process, as described herein. For example, a resection device of the present disclosure may comprise an energy-based arrangement capable of penetrating tissue towards a target lesion 1320. In one embodiment depicted in FIG. 1, tissue resection device 1100 includes an outer tube 1105 is provided having a distal edge profile and having an inner diameter IDouter. A coil 1110 is attached to the outer tube 1105 where the coil turns are spaced from and opposed to a distal end of the outer tube 1105. The coil 1110 preferably has a slightly blunted tip 1115 to minimize the possibility that it will penetrate through a blood vessel while being sufficiently sharp to penetrate tissue such as pleura and parenchyma. In some embodiments, the coil 1110 may take the form of a helix having a constant or variable pitch. The coil 1110 may also have a variable cross-sectional geometry. A coil electrode 1130 is disposed on a surface or embedded within the coil 1110.

In some embodiments, as illustrated in FIG. 1, the coil 1110 may include a plurality of contiguous coil segments, e.g., coil segments 1120 and 1125. Coil segment 1120 comprises a helical member having a pitch of zero, e.g., a generally planar open ring structure, having an inner diameter IDcoil and an outer diameter ODcoil. Coil segment 1125 comprises a helical structure of constant or variable pitch and constant or variable cross-sectional geometry. In this embodiment, the coil electrode 1130 may be disposed on a surface of or embedded in coil segment 1120.

A central tube 1200 is provided having a distal end with an edge profile comprising one or more surface segments and having an outer diameter ODcentral and an inner diameter IDcentral. As illustrated in FIG. 2, an anvil electrode 1205 is disposed on or embedded within at least one of the surface segments. The central tube 1200 is slidably disposed within the outer tube 1105 and positioned such that anvil electrode 1205 opposes and overlaps at least a portion of coil electrode 1130. The space between anvil electrode 1205 and coil electrode 1130 is referred to as the tissue clamping zone 1140. In keeping with an aspect of the present disclosure, ODcentral>IDcoil and ODcoil>IDcentral. In some embodiments, ODcentral is about equal to ODcoil. Accordingly, the central tube 1200 may be advanced through the tissue clamping zone 1140 towards the coil 1110 such that anvil electrode 1205 abuts coil electrode 1130.

A cutting tube 1300 is slidably disposed within central tube 1200. The distal end of the cutting tube 1300 is provided with a knife edge 1302 to facilitate tissue cutting.

To enable tissue resection, the resection device 1100 may be inserted into tissue and the outer tube 1105 may be advanced a predetermined distance towards a target. Coil segment 1125 allows the device to penetrate the tissue in a manner similar to a corkscrew. As coil segment 1125 penetrates tissue, any vessel in its path is either moved planar to the coil segment 1120 or pushed away from the coil 1100 for subsequent turns. The coil tip 1115 is made blunt enough to minimize chances that it will penetrate through a blood vessel while still sharp enough to penetrate certain tissue such as the lung pleura and parenchyma. The central tube 1200 may then be advanced a predetermined distance towards the target. Any vessels that are disposed in the tissue clamping zone 1140 will be clamped between coil electrode 1130 and anvil electrode 1205. The vessels can then be sealed by the application of bipolar energy to coil electrode 1130 and anvil electrode 1205. Once blood vessels are sealed, the cutting tube 1300 is advanced to core the tissue to the depth that the outer tube 1105 has reached. The sealing and cutting process can be repeated to create a core of desired size.

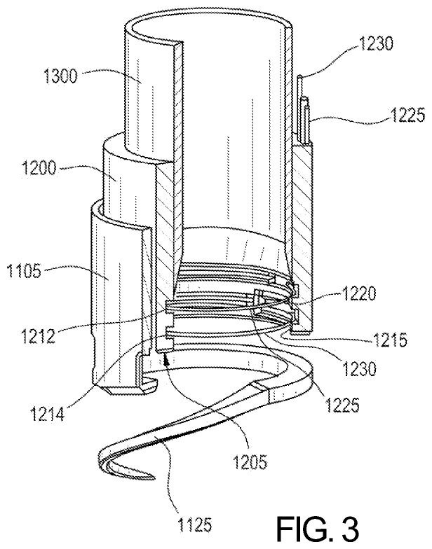

In keeping with an aspect of the present disclosure, the resection device 1100 may be further configured to dissect a target lesion 1320 and seal tissue proximate the dissection point. To facilitate dissection and sealing, as illustrated in FIG. 3, the central tube 1200 is provided with a ligation snare 1230, a first ligation electrode 1215, a second ligation electrode 1220, and an amputation snare 1225. As used herein, the word “snare” refers to a flexible line, e.g., a string or a wire. The inner wall surface of the central tube 1200 includes upper and lower circumferential grooved pathways 1212 and 1214 disposed proximate the distal end. The first and second ligation electrodes 1215 and 1220 are disposed on the inner wall of the central tube 1200 such that lower circumferential groove 1214 is between them. Upper grooved pathway 1212 is disposed axially above the ligation electrodes 1215 and 1220.

The ligation snare 1230 is disposed in lower circumferential groove 1214 and extends through the central tube 1200 and axially along the outer wall surface to a snare activation mechanism (not shown). Amputation snare 1225 is disposed in upper circumferential groove 1212 and extends through the central tube 1200 and axially along the outer wall surface to a snare activation mechanism (not shown). The outer surface of the central tube 1200 may be provided with a plurality of axially extending grooved pathways which receive the amputation snare 1225, the ligation snare 1230 and are in communication with upper and lower circumferential grooved pathways 1212 and 1214. In addition, electrode leads for the ligation electrodes 1215 and 1220 may extend to an energy source via the axially extending grooved pathways.

In operation, the resection device 1100 of this embodiment can detach and seal the tissue core. The cutting tube 1300 may be retracted to expose the ligation snare 1230 which is preferably made of flexible line, e.g., suture. The ligation snare 1230 may be engaged to snag tissue and pull tissue against the inner wall surface between the first and second ligation electrodes 1215 and 1220. Bipolar energy is then applied to first and second ligation electrodes 1215 and 1220 to seal, i.e., cauterize, the tissue. Once sealed, the cutting tube 1300 may be further retracted to expose the amputation snare 1225 which may then be activated to sever the tissue core upstream from the point where the tissue was sealed (ligation point). In some embodiments, the amputation snare 1225 has a smaller diameter than that of the ligation snare 1230. The smaller diameter facilitates tissue slicing. Accordingly, the resection device 1100 according to this embodiment both creates a tissue core and disengages the core from surrounding tissue.

In an alternative embodiment, the resection device 1100 of the present disclosure is provided with a single snare disposed between the ligation electrodes which both ligates and cuts tissue. In this embodiment, the single snare first pulls tissue against the inner wall surface of the central tube 1200 between the ligation electrodes 1215 and 1220. Bipolar energy is then applied to the first and second ligation electrodes 1215 and 1220 to seal, i.e., cauterize, the tissue. Once sealed, the snare is further pulled to sever the tissue core.

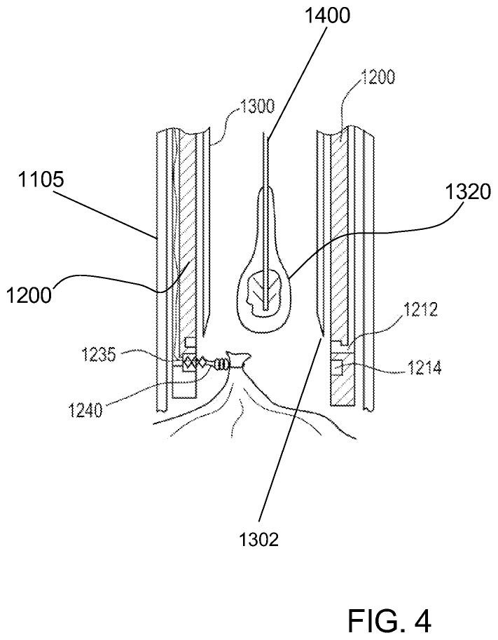

In yet another embodiment, cutting and sealing may be performed without employing electrodes. In this embodiment, the ligation snare 1230 includes a set of knots 1235 and 1240 which tighten under load, shown, for example, in FIG. 4. Ligation is performed by retracting the cutting tube 1300 to expose the ligation snare 1230 and activating the ligation snare 1230 which lassos tissue as the ligation knot tightens. Once the tissue is lassoed, the cutting tube 1300 may be further retracted to expose amputation snare 1225 which may then be activated to sever the tissue core upstream from the point where the point where the tissue was lassoed.

The present disclosure also contemplates a method and system for using the resection device to remove tissue lesions, for example, lung lesions. The method generally comprises anchoring the lesion targeted for removal, creating a channel in the tissue leading to the target lesion 1320, creating a tissue core which includes the anchored lesion, ligating the tissue core and sealing the surrounding tissue, and removing the tissue core including the target lesion 1320 from the channel.

Anchoring may be performed by any suitable structure for securing the device to the lung. Once the lesion is anchored, a channel may be created to facilitate insertion of the resection device 1100. The channel may be created by making an incision in the lung area and inserting a tissue dilator and port into the incision. A tissue core which includes the anchored lesion may be created. In keeping with the present disclosure, the resection device 1100 may be used to create the tissue core, to ligate the tissue core and to seal the tissue core and sever it from the surrounding tissue as described hereinabove. The tissue core may then be removed from the channel. As an example, a cavity port may be inserted in the channel to facilitate subsequent treatment of the target lesion 1320 site through chemotherapy and/or energy-based tumor extirpation such as radiation. As a further example, a cavity port may be disposed on the perimeter of the tissue resection apparatus. When the apparatus is removed from the tissue site, the cavity port may remain in place or may be removed.

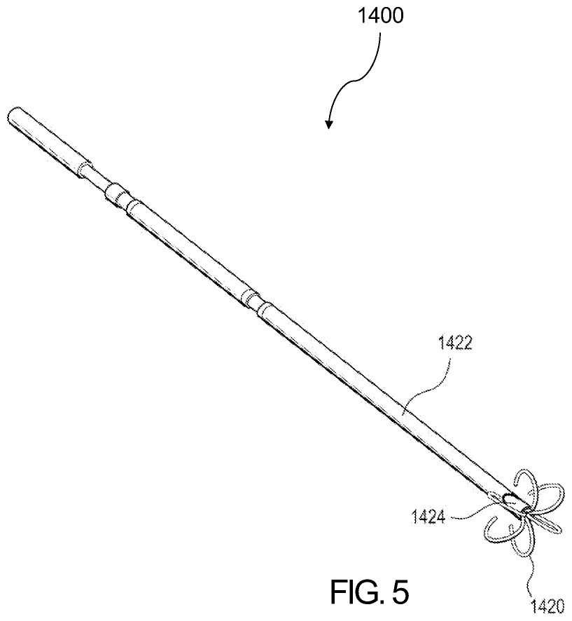

The anchor 1400 depicted in FIG. 5 is suitable for use in performing the method for removing tissue lesions described herein. The anchor 1400 comprises an outer tube 1422 having a sufficiently sharp edge to pierce the chest cavity tissue and lung without causing excess damage and an inner tube 1424 disposed within the outer tube 1422. One or more tines or fingers 1420 formed from shape memory material, e.g., Nitinol, preformed are attached to the end of the inner tube 1424. The outer tube 1422 is retractably disposed over the inner tube 1424 such that when the outer tube 1422 is retracted, the tines 1420 assume their preform shape as shown. In keeping with the present disclosure, the outer tube 1422 is retracted after it has pierced the lung lesion thereby causing the tines 1420 to engage the lung lesion. Other suitable anchors may include coils and suction-based structures.

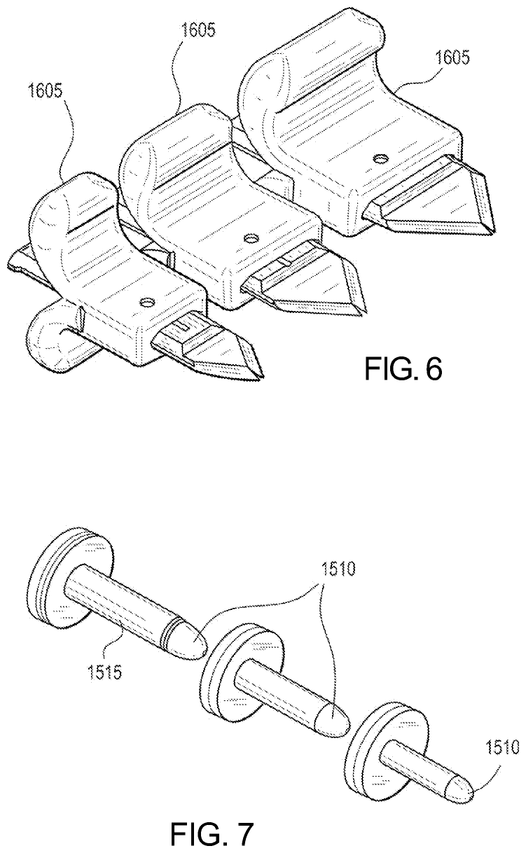

The incision blades depicted in FIG. 6 are suitable for use in performing the method for removing tissue lesions described herein. Once the anchor 1400 is set, it is preferable to create a small cut or incision to facilitate insertion of chest wall tissue dilator. Incision blades 1605 are used to make a wider cut. The incision blades 1605 may be successive and may include a central aperture which allows them to be coaxially advanced along the anchor 1400 to create a wider cut in the chest wall, with each successive blade being larger than the previous blade, thereby increasing the width of the incision.

The tissue dilator depicted in FIG. 7 is suitable for use in performing the method for removing tissue lesions described herein. The tissue dilator may comprise any suitable device for creating a channel in organic tissue. In one exemplary embodiment, the tissue dilator assembly includes a single cylindrical rod with a rounded end 1510 or a cylindrical rod with rounded end 1510 and a rigid sleeve arrangement 1515. Successive tissue dilators are coaxially advanced along the anchor to create tissue tract or channel in the chest wall, with each successive dilator being larger than the previous dilator, thereby increasing the diameter of the channel. Once the final dilator with rigid sleeve is deployed, the inner rod is removed while leaving the rigid sleeve in the intercostal space between ribs to create direct passage to the lung pleura.

Any tissue resection device capable of penetrating lung tissue and creating a tissue core including a target lesion 1320 is suitable for use in performing the method for removing tissue lesions described herein. The tissue resection device 1100 described herein is preferred.

Once the tissue resection device 1100 is removed, a small channel in the lung exists where the target lesion was removed. This channel may be utilized to introduce an energy-based ablation device and/or localized chemotherapy depending on the results of the tissue diagnosis. Accordingly, the method and system of the present disclosure may not only be utilized to ensure an effective biopsy is performed but also complete removal of the lesion with minimal healthy lung tissue removal.

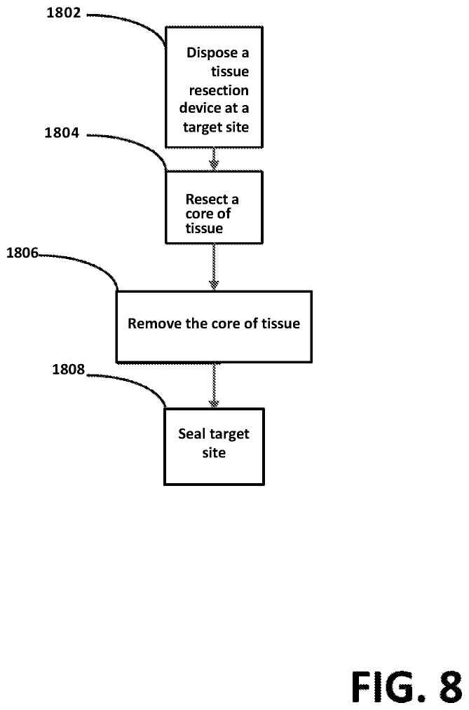

FIG. 8 shows a flow diagram of an example method. Tissue at a target site may be cored such that a tissue core is removed from the target site thereby creating a core cavity at the target site. Coring tissue at a target site may comprise transecting and sealing tissue. Coring tissue at a target site may comprise disposing a tissue coring apparatus adjacent to a target tissue site. The tissue coring apparatus may comprise a first clamping element comprising a helical coil, a second clamping element, the second clamping element being positioned to oppose at least a portion of the first clamping element, a first and second electrode configured for the delivery of radiofrequency energy for sealing tissue, and/or a cutting element configured for the transection of at least a portion of the sealed tissue. Other apparatus may be used.

At 1802, a tissue resection device may be disposed at a target tissue site. The target tissue site may comprise a tissue lesion. Various tissues may be comprised as the target site. The tissue resection device may comprise one or more of the devices or components described herein.

At 1804, a core of tissue may be resected. A core of tissue may have a prescribed (e.g., pre-defined) shape (e.g., columnar) and dimension based on a coring apparatus. Such coring apparatus may be used to core the same or substantially the same shaped tissue core in a repeatable manner. Such coring may be distinguished from other tissue removal, for example using scissors or scalpel, where the cut tissue will not have a pre-defined shape or dimensions. As an example, the tissue resection device may be caused to resect a core of tissue from the target tissue site.

At 1806, the tissue resection device may be removed from that body or create a core cavity at the target tissue site. Since a core of tissue was removed, biological fluid may flow toward or into the core cavity.

At 1808, at least a portion of the core cavity may be sealed. Such sealing may comprise sealing biological fluid vessels. The sealing biological fluid vessels may minimize flow of biological fluids into the cavity core.

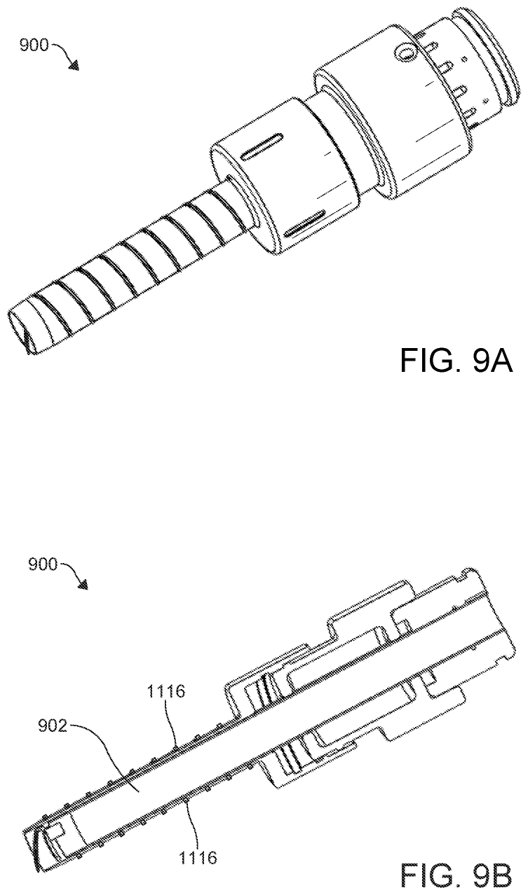

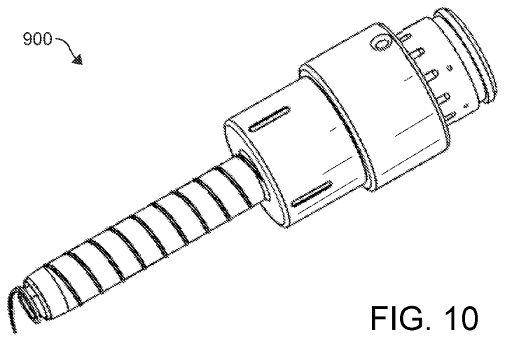

As described herein, access to a target tissue site can be achieved via a trocar 900. Example trocars are shown in FIGS. 9-10. Trocars may comprise a trocar channel (e.g., trocar channel 902 of FIG. 9B). The trocar channel may be used to allow air to be introduced into the pleural space when the first layer of the pleural space is penetrated. The intrapleural vacuum is lost, and thus the lung is dropped away to minimize the potential of damaging to the lung pleura. Once a lesion has been successfully located, an anchoring device can be used to stabilize the target tissue lesion. The tissue coring device can also be introduced directly to the location of the target lesion using a trocar or under direct visualization with or without a guide anchor and perform the tissue resection. The outer tube 1105 may have protrusions 1116 such as ribs, villi, and the like on the outer surface to better hold the trocar 900 in place during use.

A coring device may be configured to core and amputate the target tissue, as shown and described herein. Additionally or alternatively, a coring device and an amputation device may be configured as discrete devices. The coring device may be used to perform coring and it is removed leaving the anchor 1400 and tissue cored attached to surrounding tissue at the bottom of the tissue cavity. A user may perform visual inspection of the sample prior to deploying an amputation device onto the anchor 1400 to amputate and then remove the tissue sample for subsequent tissue analysis.

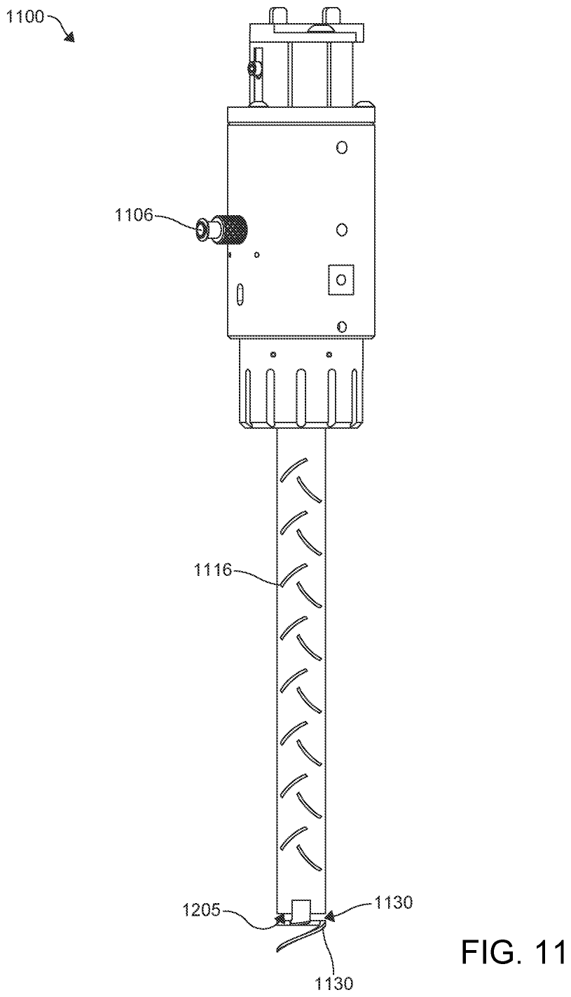

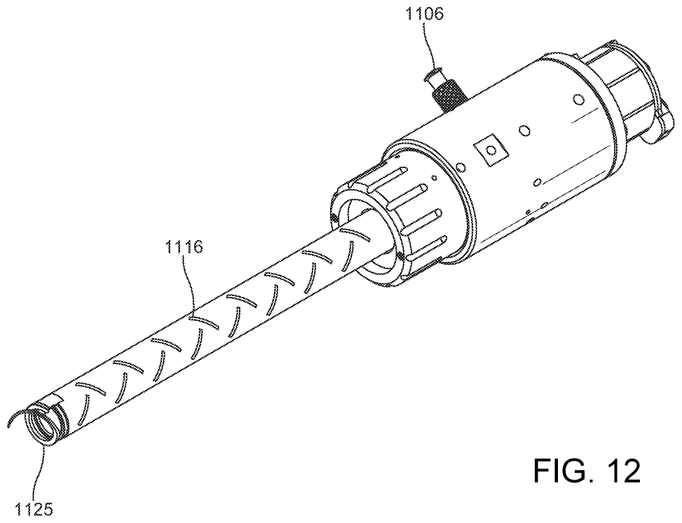

As shown, for example, in FIGS. 11-12, a bipolar coring device 1100 may be configured to core a target lesion percutaneously. As shown, a coil electrode 1130 at the distal tip is one pole and the anvil ring electrode 1205 on the distal tip of the central tube 1200 is the second pole. Protrusions 1116 on the outer surface of the outer tube 1105 aid in fixing the device 1100 in place. The luer port 1106 on the handle is for vacuum connection during coring. In use, a user places the device 1100 over the anchor 1400 and may perform coring until the anchor 1400 is locked onto the distal end of the device. At this point, the target lesion 1320 is within the inner diameter (ID) of the cutting tube 1300 (the innermost tube). The user may unlock the anchor 1400 from the device and rotate the coil electrode 1130 counterclockwise to disengage it from the surrounding tissue before removing the device 1100 from the anchor. The target lesion 1320 and anchor 1400 may be left in place. A user may visually inspect the target lesion 1320 while it is still attached to the lung tissue.

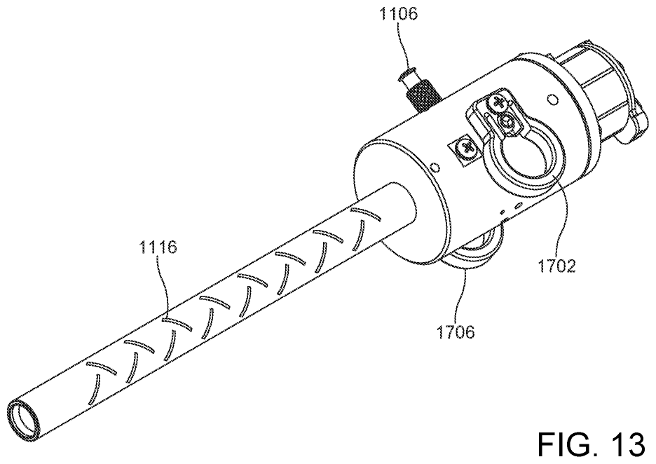

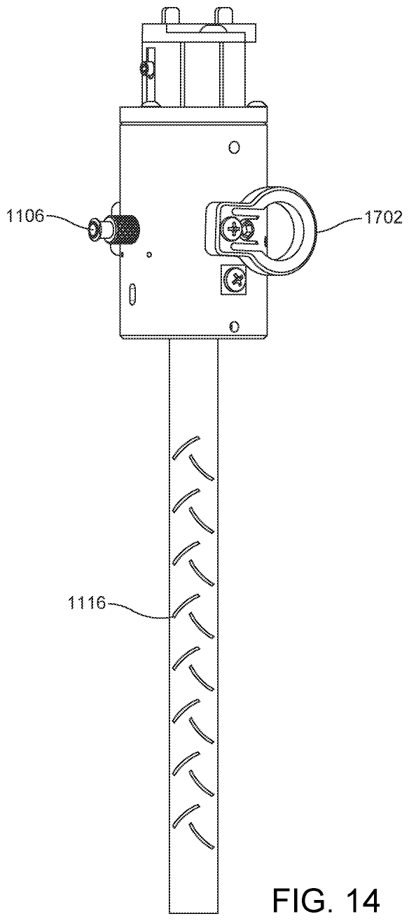

As shown, for example, in FIGS. 13-14, a bipolar device 1100 may be configured to ligate and amputate a cored target lesion 1320 from surrounding tissue. As shown, two flexible lines 1225, 1230 may be disposed in the internal grooves 1212, 1214 on the ID of the central tube 1200. The ligation line groove 1214 is placed between the two ligation electrodes 1215, 1220. The amputation line groove 1212 is placed proximal to the two electrodes. The luer port 1106 on the handle is for vacuum connection during use. In an example use, post coring, the device 1100 is inserted over the anchor 1400 until the anchor 1400 is locked at the distal end of the device. The target lesion 1320 is within the inner diameter of the device 1100 once the anchor 1400 is locked in place. The cutting tube 1300 is then moved back to expose the ligation line 1230 and the amputation line 1225. The ligation ring 1706 on the handle is activated to pull tissue distal to the lesion against the two electrodes for ligation. Once ligation is completed, the amputation ring 1702 on the handle is pulled to amputate tissue between the target lesion 1320 and ligation location to disconnect the lesion from lung tissue. The device with the target lesion 1320 and anchor 1400 is then removed for lesion tissue collection. The action of pulling manually on either of the rings 1702, 1706 can be designed to use an internal mechanism, such as motor, spring loaded, cam action, etc.

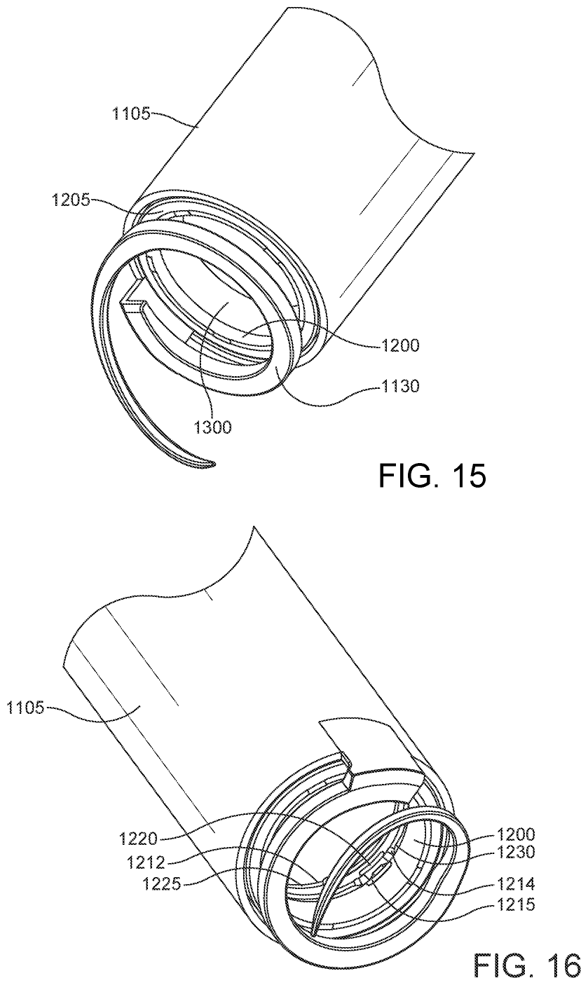

As shown, for example, in FIGS. 15-16, an embodiment of the tissue resection device 1100 may comprise an outer tube 1105, a central tube 1200, and a cutting tube 1300. At the distal end of the central tube 1200, there may be an anvil electrode 1205. On the coil there may be a coil electrode 1130. The region between the anvil electrode 1205 and the coil electrode 1130 is referred to as the tissue clamping zone 1140. FIG. 15 depicts the structure when the cutting tube 1300 is withdrawn further within the central tube 1200. The first ligation electrode 1215 and the second ligation electrode 1220 are disposed on the inner wall of the central tube 1200 such that lower circumferential groove 1214 containing the ligation snare 1230 is between them. An upper grooved pathway 1212 is disposed axially above the ligation electrodes 1215 and 1220 for the amputation snare 1225.

The present disclosure comprises at least the following aspects:

Aspect 1. A tissue coring and amputating system comprising: a coring device comprising: a first clamping element comprising a helical coil, a second clamping element, the second clamping element being positioned to oppose at least a portion of the first clamping element, a first electrode and a second electrode configured for the delivery of radiofrequency energy to an area adjacent one or more of the first clamping element and the second clamping element to seal tissue, and a cutting element configured for the transection of at least a portion of the sealed tissue to form a core of tissue; and an amputating device comprising: a second cutting element configured for the amputation of at least the core of tissue.

Aspect 2. A method of using the tissue coring and amputating system of aspect 1, the method comprising: disposing the coring device adjacent the target tissue site; rotating the helical coil into the target tissue site; causing the first clamping element and the second clamping element to clamp against each other; causing radiofrequency energy to energize one or more of the first electrode and the second electrode; causing the cutting element to core at least a portion of the target tissue site; removing the coring device and a core of tissue; disposing the amputation device adjacent the target tissue site; and causing the amputation device to amputate at least a portion of the target tissue site.

Aspect 3. The tissue coring and amputating system of any one of aspects 1-2, further comprising: a first actuator operable to actuate the first or second clamping element to apply mechanical compression to tissue; and a second actuator operable to actuate the cutting element to transect tissue.

Aspect 4. The tissue coring and amputating system of any one of aspects 1-3, wherein the helical coil comprises first and second contiguous coil segments

Aspect 5. The tissue coring and amputating system of aspect 4, wherein the first coil segment comprises a generally planar open ring

Aspect 6. The tissue coring and amputating system of aspect 5, wherein the first coil segment is helical and has a pitch of zero.

Aspect 7. The tissue coring and amputating system of any one of aspects 1-4, wherein the second coil segment is helical and has a non-zero pitch.

Aspect 8. The tissue coring and amputating system of aspect 7, wherein the second coil segment has a variable pitch.

Aspect 9. The tissue coring and amputating system of any one of aspects 1-4, wherein the first coil segment is helical and has a first pitch and the second coil segment is helical and has a second pitch, and at least one of the first and second pitches is variable.

Aspect 10. The tissue coring and amputating system of any one of aspects 1-9, wherein the first electrode is provided by at least a portion of the first clamping element.

Aspect 11. The tissue coring and amputating system of any one of aspects 1-10, wherein the second electrode is provided by at least a portion of the second clamping element.

Aspect 12. The tissue coring and amputating system of any one of aspects 1-11, wherein the helical coil includes a blunt tip.

Aspect 13. The tissue coring and amputating system of any one of aspects 1-12, wherein the first and second electrodes have surface profiles that are substantially matching.

Aspect 14. The tissue coring and amputating system of any one of aspects 1-13, wherein at least a portion of the cutting element comprises a sharpened edge.

Aspect 15. The tissue coring and amputating system of any one of aspects 1-14, wherein the cutting element comprises at least one electrode configured for the delivery of radiofrequency energy.

Aspect 16. The tissue coring and amputating system of any one of aspects 1-15, wherein the cutting element comprises an ultrasonic blade.

Aspect 17. The tissue coring and amputating system of any one of aspects 1-16, wherein at least a portion of the second cutting element comprises a sharpened edge.

Aspect 18. The tissue coring and amputating system of any one of aspects 1-17, wherein the second cutting element comprises at least one electrode configured for the delivery of radiofrequency energy.

Aspect 19. The tissue coring and amputating system of any one of aspects 1-18, wherein the second cutting element comprises an energized wire.

Aspect 20. The tissue coring and amputating system of any one of aspects 1-19, wherein the second cutting element comprises a suture.

Aspect 21. The tissue coring and amputating system of any one of aspects 1-20, further comprising an actuator operable to actuate the second cutting element to transect tissue.

Although shown and described is what is believed to be the most practical and preferred embodiments, it is apparent that departures from specific designs and methods described and shown will suggest themselves to those skilled in the art and may be used without departing from the spirit and scope of the invention. For example, the systems, devices, and methods described herein for removal of lesions from the lung. It will be appreciated by the skilled artisan that the devices and methods described herein may are not limited to the lung and could be used for tissue resection and lesion removal in other areas of the body. The present invention is not restricted to the particular constructions described and illustrated, but should be constructed to cohere with all modifications that may fall within the scope of the appended claims.

What is claimed is:

1. A tissue coring and amputating system comprising:
a coring device comprising:
a first clamping element comprising a helical coil,
a second clamping element, the second clamping element being positioned to oppose at least a portion of the first clamping element,
a first electrode and a second electrode configured for the delivery of radiofrequency energy to an area adjacent one or more of the first clamping element and the second clamping element to seal tissue, and
a cutting element configured for the transection of at least a portion of the sealed tissue to form a core of tissue; and
an amputating device comprising:
a second cutting element configured for the amputation of at least the core of tissue.

2. A method of using the tissue coring and amputating system of claim 1, the method comprising:
disposing the coring device adjacent a target tissue site;
rotating the helical coil into the target tissue site;
causing the first clamping element and the second clamping element to clamp against each other;
causing radiofrequency energy to energize one or more of the first electrode and the second electrode;
causing the cutting element to core at least a portion of the target tissue site;
removing the coring device and a core of tissue;
disposing the amputation device adjacent the target tissue site; and
causing the amputation device to amputate at least a portion of the target tissue site.

3. The tissue coring and amputating system of claim 1, further comprising:
a first actuator operable to actuate the first or second clamping element to apply mechanical compression to tissue; and
a second actuator operable to actuate the cutting element to transect tissue.

4. The tissue coring and amputating system of claim 1, wherein the helical coil comprises first and second contiguous coil segments.

5. The tissue coring and amputating system of claim 4, wherein the first coil segment comprises a generally planar open ring.

6. The tissue coring and amputating system of claim 5, wherein the first coil segment is helical and has a pitch of zero.

7. The tissue coring and amputating system of claim 4, wherein the second coil segment is helical and has a non-zero pitch.

8. The tissue coring and amputating system of claim 7, wherein the second coil segment has a variable pitch.

9. The tissue coring and amputating system of claim 4, wherein the first coil segment is helical and has a first pitch and the second coil segment is helical and has a second pitch, and at least one of the first and second pitches is variable.

10. The tissue coring and amputating system of claim 1, wherein the first electrode is provided by at least a portion of the first clamping element.

11. The tissue coring and amputating system of claim 1, wherein the second electrode is provided by at least a portion of the second clamping element.

12. The tissue coring and amputating system of claim 1, wherein the helical coil includes a blunt tip.

13. The tissue coring and amputating system of claim 1, wherein the first and second electrodes have surface profiles that are substantially matching.

14. The tissue coring and amputating system of claim 1, wherein at least a portion of the cutting element comprises a sharpened edge.

15. The tissue coring and amputating system of claim 1, wherein the cutting element comprises at least one electrode configured for the delivery of radiofrequency energy.

16. The tissue coring and amputating system of claim 1, wherein the cutting element comprises an ultrasonic blade.

17. The tissue coring and amputating system of claim 1, wherein at least a portion of the second cutting element comprises a sharpened edge.

18. The tissue coring and amputating system of claim 1, wherein the second cutting element comprises at least one electrode configured for the delivery of radiofrequency energy.

19. The tissue coring and amputating system of claim 1, wherein the second cutting element comprises an energized wire.

20. The tissue coring and amputating system of claim 1, wherein the second cutting element comprises a suture.

21. The tissue coring and amputating system of claim 1, further comprising an actuator operable to actuate the second cutting element to transect tissue.
